(19) United States
(12) Patent Application Publication
(10) Pub. No.: US 2026/0120851 A1
(43) Pub. Date: Apr. 30, 2026

(54) METHOD AND SYSTEM FOR OPTIMIZING PROVISION OF MEDICAL IMAGE DATA

(71) Applicant: Siemens Healthineers AG, Forchheim (DE)

(72) Inventor: Alois Regensburger, Poxdorf (DE)

(21) Appl. No.: 19/369,222

(22) Filed: Oct. 25, 2025

(30) Foreign Application Priority Data
Oct. 25, 2024 (DE) 10 2024 210 305.5

(51) Int. Cl. G16H 30/40; G06V 10/25; G16H 30/20

(52) U.S. Cl. CPC G16H 30/40; G06V 10/25; G16H 30/20

(57) ABSTRACT

A method for optimizing provision of medical image data includes providing medical image data of an examination object from a provision unit to a processing unit. The method includes processing of the medical image data by the processing unit, providing a signal from the processing unit to the provision unit when a predefined processing state of the medical image data is reached, and providing the processed medical image data from the processing unit to a receive unit. On receipt of the signal by the provision unit, the providing of the medical image data, the processing, the providing of the signal, and the providing of the processed medical image data are executed repeatedly.

### BACKGROUND

This application claims the benefit of German Patent Application No. DE 10 2024 210 305.5, filed on Oct. 25, 2024, which is hereby incorporated by reference in its entirety.

The present embodiments relate to a method for optimizing provision of medical image data, to a system for optimizing provision of medical image data, and to a computer program product.

Medical image data may be captured and processed almost in real time by modern imaging technology. This may be of great importance particularly for robotic interventions, since the aim is an image-based control of the movements of the robot. A small time delay (e.g., a low latency time) between recording an image and a reaction of the robot is often relevant for these applications. With remote-controlled robot interventions, for example, during which an increased overall time delay occurs, this may make moving the robot under image control in real time more difficult.

Previous solutions to solving this problem include a fixed image refresh rate during image recording. Should an image processing be slower than the refresh rate of the image recording, a queue of image data to be processed begins to form. In order to avoid a further latency buildup, image data is often removed from the queue. As an alternative, processing systems with deterministic run times (e.g., Field Programmable Gate Arrays (FPGA)) or with run times as slow as possible are employed in order to make possible a performance buffering, which, however, comes with a high cost overhead.

Despite these advancements and solutions, challenges still remain (e.g., with a non-deterministic execution of image processing tasks on GPUs and CPUs), which may lead to fluctuations in the processing times and thereby to higher average latency times.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved processing of medical image data is provided.

Independent of the grammatical term usage, individuals with male, female, or other gender identities are included within the term.

In a first aspect, the present embodiments relate to a method for optimizing provision of medical image data. In this method, in a first act a), medical image data of an examination object is provided by a provision unit to a processing unit. Further, in a further act b.1), the medical image data is processed by a processing unit. Further, in a further act b.2), a signal is provided by the processing unit to the provision unit when a predefined processing state of the medical image data is reached. Further, in a further act c), the processed medical image data is provided by the processing unit to a receive unit. The acts a) to c) are carried out repeatedly (e.g., precisely when the signal is received by the provision unit).

In one embodiment, the acts a) to c) may be carried out at least partly at the same time or after one another. For example, act b.2) may be carried out before act b.1) has ended.

The examination object may be a human and/or animal female patient and/or a human and/or animal male patient, for example, and/or an examination phantom.

The provision of the medical image data in act a) may, for example, include a capture and/or reading out of a computer-readable data memory and/or a receipt from a data memory unit (e.g., a database). In this case, the medical image data may have been recorded by a medical imaging device. The medical image data may further be provided by the provision unit of a medical imaging device for recording the image dataset. In this act, the medical image data is transmitted by the provision unit to the processing unit. The provision unit and the processing unit may have respective interfaces for this.

The medical image data may include a representation (e.g., a depiction and/or a model) of the examination object. In this case, the medical image data may represent the examination object spatially resolved in two dimensions (2D) and/or three dimensions (3D) (e.g., depict it and/or model it). Further, the medical image data may be time-resolved (e.g., depict a scene). The image data may have a number of image points (e.g., pixels and/or voxels), each with at least one image value (e.g., a number of image values, such as time intensity curves). In this case, the image values of the image points each represent a part volume of the examination object (e.g., depict it and/or model it).

The processing of the image data in act b.1) may include an application of at least one processing step (e.g., of a number of processing step) to the medical image data. For example, the processing of the image data may include an application of at least one processing function (e.g., of a number of processing functions) to the medical image data. The processing of the medical image data may, for example, include a noise suppression, segmentation, temporal and/or spatial filtering, feature extraction, registration (e.g., to another image data set and/or one recorded at a previous time), movement compensation, and/or amplification. The processing unit may employ algorithms such as machine learning and artificial intelligence in the processing of the medical image data in order to process the medical image data to analyze it and/or to extract relevant information.

The processing unit may include one or more processors (e.g., Central Processing Unit (CPU)) and/or graphics processors (e.g., Graphics Processing Unit (GPU)) that are configured to execute complex algorithms for image processing. In a further form of embodiment, the processing unit may also include specialized hardware units (e.g., a Field Programmable Gate Array (FPGA)) that are optimized for specific processing steps.

As soon as a predefined processing stage of the processing of the medical image data is reached, the processing unit in act b.2) may provide the signal to the provision unit (e.g., send it). The provision of the signal by the processing unit to the provision unit may be undertaken via respective interfaces. The signal may include information about the processing stage of the medical image data at that moment. The signal may further serve as a trigger for the provision unit to provide further medical image data. Reaching of the predefined processing state may be identified by a checkpoint and/or a means for software evaluation. In this case, based on the reaching of the predefined processing state (e.g., the checkpoint), a time remaining until the start of a repeated processing of medical image data may be able to be predicted. The predefined processing stage may, for example, be characterized by reaching or ending of a predefined processing step of a number of processing steps for processing the medical image data. As an alternative or in addition, the predefined processing stage may be characterized by a predefined proportion of processing (e.g., of a processing progress) of the medical image data. The proportion may lie between 0 and 100 % (e.g., below 100%) of a final processing state of the medical image data. For example, the predefined processing stage may be different from a final processing stage of the medical image data. A period of time between the provision of the signal and the repeated provision of the medical image data to the processing unit (e.g., an insertion of the image data into an input queue of the processing unit) may be known and deterministic. In this case, a remote transmission in the provision of the image data to the processing unit may represent an exception to the deterministic period of time. A latency time at that point in the remote transmission over a remote transmission channel (e.g., a network and/or Internet) is to be taken into account.

The provision of the processed medical image data by the processing unit to the receive unit may include a transmission of the processed medical image data (e.g., using a further signal) to the receive unit. The processing unit and the processing unit may each have interfaces for this. The receive unit may include a receive unit of a robotic control, where the robotic control is based on the processed medical image data. As an alternative or in addition, the receive unit may include a display unit for display of a graphical representation of the processed medical image data. The display unit may, for example, include a monitor and/or a display and/or a projector and/or smart glasses. For example, depending on the processed medical image data provided, a control signal for controlling a device (e.g., a medical device, such as a robotic control) may be provided. In one embodiment, in act c), a graphical representation of the processed medical image data may be provided by the display unit (e.g., displayed). This may be shown in the form of 2D or 3D images that are displayed on a display surface of the display unit.

In one embodiment, the acts a) to c) (e.g., the acts a), b.1), b.2) and c)) may be carried out repeatedly if the signal is available (e.g., present). For example, the acts a) to c) (e.g., the acts a), b.1), b.2) and c)) are carried out repeatedly by the provision unit on receipt of the signal. In one embodiment, the acts a) to c) are carried out until an abort condition occurs and are carried out repeatedly if the signal is present. In this case, the abort condition may predetermine a maximum number of repetitions and/or a maximum duration for carrying out the repetitions. The abort condition may further be activated (e.g., occur) through a further (e.g., external) signal and/or through a user input.

The method of the present embodiments may make possible lower and more stable latency times during the processing of medical image data. A higher efficiency may further be achieved in the use of hardware resources, since less need exists for overdimensioned CPU and GPU specifications. Further, cost savings may be made possible by employing standard hardware components. Further, the method of the present embodiments may make possible an optimized image processing in real time, which is of great importance, especially for robotic interventions.

The GPU and/or CPU of the processing unit may have non-deterministic run times (e.g., due to different loads for other processing tasks and/or because a few algorithms may have runtime-dependent or context-dependent runtimes or computing loads).

In summary, the method for optimizing provision of medical image data offers an efficient and deterministic solution for minimizing the latency times and for optimizing the image processing in real time. This is advantageous for robotic interventions and other medical applications in which a fast and precise processing of the image data is to be provided.

In a further form of embodiment, the method act a) may include recording of the medical image data using a medical imaging device. The medical imaging device may include the provision unit.

The medical imaging device may, for example, include a Magnetic Resonance Tomograph (MRT) and/or a Computed Tomography (CT) system and/or a medical x-ray device (e.g., a medical C-arm x-ray device), and/or an ultrasound device, and/or a Positron Emission Tomography (PET) device, and/or an optical camera (e.g., in an endoscope and/or an operation microscope and/or an optical coherence tomography (OCT)).

In one embodiment, the medical image data may be recorded by the medical imaging device. In one embodiment, the recording of the medical image data may be undertaken by the medical imaging device depending on the signal (e.g., depending on the presence of the signal). In this case, the medical image data may be repeated using the medical imaging device (e.g., recorded in each case when the signal is present).

The fact that the recording of the medical image data by the medical imaging device occurs depending on the signal (e.g., on receipt of the signal by the provision unit) enables a load on the examination object during the recording of the image data (e.g., by an x-ray dose) to be minimized. The fact that only image data is recorded that is able to be processed by the processing unit enables an overheating and/or an increased need for energy and/or battery use of the medical imaging device to be minimized. An overheating and/or an increased need for energy and/or battery use of the medical imaging device may be caused, for example, by an image recording frequency that lasts too long and/or is constant. Using the method of the presnet embodiments, the recording of the medical image data (e.g., an x-ray image recording) may be initiated at the last possible moment and a latency time minimized for a varying processing time for each item of image data.

In a further form of embodiment of the method, a future provision time for repeated carrying out of act c) may be identified. The signal in act b.2) may additionally be provided depending on the future provision time.

The future provision time may specify a point in time located in the future at which a repeated (e.g., a next) iteration of act c) is to be carried out. The identification of the future provision time may be undertaken manually or automatically (e.g., based on the medical image data and/or a latency time). The identification of the future provision time may include a determination, capture, establishment, estimation, and/or measurement of the future provision time.

In one embodiment, the provision of the signal in act b.2) may additionally be undertaken depending on the future provision time. In this case, the signal having information about the future provision time may be provided. As an alternative or in addition, the provision of the signal may be undertaken after a delay or immediately depending on the future provision time. For example, in the provision of the signal depending on the future provision time, a latency time between the processing unit and the provision unit, a provision duration of the provision unit for provision of the medical image data, a processing duration of the processing unit for processing the medical image data, and/or a latency time between the processing unit and the receive unit may be taken into account.

Using the form of embodiment, it may be provided that the graphical representation of the processed image data may occur at the future provision time.

In a further form of embodiment of the method, the future provision time may be identified based on: a feature that is identified in the medical image data in act b.1); a change that is identified in the medical image data in act b.1); a latency time of a robotic control (e.g., of a robotic control of a medical object on or in the examination object); or any combination thereof. The robotic control may be based on a graphical representation of the processed image data.

The feature may include an anatomical and/or geometrical feature of the examination object and/or of a medical object that is arranged on or in the examination object. Geometrical features may, for example, include a contour and/or a contrast and/or a marker structure. Anatomical features may, for example, include a tissue contrast and/or a tissue boundary. The identification of the feature (e.g., of a number of features) in the medical image data may, for example, include a segmentation and/or pattern recognition.

For example, a degree of contrasting and/or visibility of an anatomical and/or medical object in the medical image data may influence a run time of detection algorithms for identification of a depiction of the anatomical and/or medical object. The change that is depicted in the image data (e.g., a momentary movement of the examination object, such as a heartbeat and/or a breathing movement) may further change its momentary or predicted speed and/or character. This may influence the image quality of the image data (e.g., via a movement artifact). Through this, a run time of an image processing algorithm (e.g., for correction of the movement artifact and/or for detection tasks despite the movement artifact) may change. A run time of a registration algorithm and/or of a robotic control algorithm may also be influenced by a speed of the change at that moment (e.g., a speed of movement, such as depending on a heart phase). A, for example, geometrical depth of a medical and/or anatomical object to be identified in the image data may further influence a run time of a detection algorithm. For example, an image quality of an ultrasound image of an examination region of an examination object with an object arranged therein may be influenced by an increasing depth of the arrangement of the object in the examination object (e.g., a body of the examination object). A concentration of contrast media in the examination object may further influence a run time of a detection algorithm and/or of a registration algorithm and/or of a movement compensation algorithm.

The change may include a temporal and/or spatial change in the examination object, which is depicted in the medical image data. The change may, for example, include a dynamic change on or in the examination object (e.g., a movement of at least a part of the examination object, such as of a tissue and/or organ), and/or a movement of a contrast medium, and/or a movement of a medical object in the examination object. The identification of the change in the medical image data may, for example, be based on an analysis (e.g., a comparison) of the time intensity curves of the image points of the medical image data.

In one embodiment, the identification of the change may include an identification of a temporal and/or spatial rate of change. In such cases, the future provision time may be determined such that (e.g., based on the identified change) a depiction rate during the provision of the image data at least corresponds to the temporal rate of change of the change. This enables it to be provided that the identified change in the image data is able to be depicted spatially and temporally resolved.

The robotic control may include a control of a medical device (e.g., of a medical imaging device) and/or of a medical object using a movement apparatus.

The medical object may be depicted as a surgical and/or diagnostic instrument (e.g., as a catheter and/or microcatheter and/or endoscope and/or guide wire) and/or implant. The medical object may further be robotically controllable by a, for example, robotic movement apparatus (e.g., a catheter robot). The robotic control of the medical object may include a positioning (e.g., alignment and/or deformation and/or placing) and/or movement (e.g., translating and/or rotating) of the medical object (e.g., at least one section of a medical object).

A run time of an image processing algorithm and/or detection algorithm may, for example, be influenced with the aid of a medical object, which has been detected in image data recorded beforehand. In this case, the run time may be influenced, for example, by a kind and/or a type of the detected medical object. A location and/or form of the medical object (e.g., of a robotic catheter) relative to an anatomy and/or the examination object may further influence a run time of a robotic control algorithm and/or a latency time of the robotic control.

The latency time of the robotic control may include a period of time from the provision of the processed medical image data using the processing unit to an actual actuation of the robotic control (e.g., of the movement apparatus). In this case, the robotic control (e.g., the positioning and/or movement) may be based on the graphical representation of the processed image data.

With robotic control, it may be desired to map a spatial reaction of the robotically moved medical object to a preceding control command in the image data. Depending on the latency time of the robotic control, the repeated provision of the medical image data may be undertaken as a soon as a reaction to the control command visible in the image data is forecast and/or expected.

The form of embodiment, through the identification of the future provision time, may provide that the graphical representation of the processed image data is able to be provided at the right time while taking into account the respective criteria. The form of embodiment may, for example, identify the future provision time based on a predictive dynamic model of the robotic control (e.g., of a robot reaction) and/or of the feature and/or change depicted in the image data (e.g., an anatomical movement) of the examination object. In this case, any given latency times between robotic control signals and a (e.g., mechanical) reaction of the movement apparatus to these robotic control signals may be taken into account. This enables a better performance of a robotic closed-loop control to be achieved (e.g., for rapid anatomical movements and/or complex movement apparatuses). A load on the examination object (e.g., an x-ray dose) may be reduced by the fact that, by taking into account the latency time of the robotic control, medical image data is provided repeatedly only when the movement apparatus has actually already reacted to a control signal provided in the past by the robotic control.

In a further form of embodiment of the method, a latency time between act b.1) and act c) may be captured (e.g., measured and/or predicted). The provision of the signal in act b.2) may additionally be undertaken depending on the last latency time captured in each case.

In one embodiment, the latency time may be captured as the period of time between the beginning of the execution of act b.1) (e.g., the beginning of the processing of the medical image data) and an end of the execution of act c) (e.g., the provision of the graphical representation of the processed image data). The capture of the latency time may, for example, be undertaken by the respective units (e.g., the processing unit and the receive unit) or by a further sensor. The provision of the signal in act b.2) may additionally be undertaken depending on the last latency time captured in each case. For example, the signal may be provided in act b.2), depending on the last latency time captured, such that the latency time may be taken into account (e.g., compensated for) when executing the repeated carrying out of the acts a) to c).

This provides that the carrying out of the act a) to c) may be dynamically adapted to the currently captured latency time in order to guarantee an optimal efficiency and accuracy.

In a further form of embodiment of the method, act b.1) may include a number of processing steps for processing the medical image data, which are carried out one after another. The provision of the signal in act b.2) may be undertaken as a function of the reaching or ending of a predefined processing step of the number of processing steps.

In one embodiment, act b.1) may include a number of processing steps that are at least partly (e.g., entirely) different, which are applied to the medical image data after one another in time (e.g., sequentially). In this case, respective processing results of a processing step may be provided to the respective subsequent processing step until the last processing step is reached. An order of the processing steps during the repeated carrying out of the acts a) to c) may be the same or different.

In one embodiment, the provision of the signal in act b.2) may be undertaken when a predefined processing step is reached (e.g., begins or ends, such as is concluded).

This enables a timely repetition of the acts a) to c) to be made possible.

In a further form of embodiment of the method, the processing unit may include at least one GPU and at least one CPU, which are configured in each case to carry out at least one of the number of processing steps.

The at least one CPU may be configured as a central processing unit, which is configured for carrying out computations and/or control tasks.

The at least one GPU may be configured as a specialized electronic circuit, which is configured for fast and efficient (e.g., parallel) processing of the medical image data. Further, the at least one GPU may have a high bandwidth and speed during data transmission. The at least one GPU may further have special memory chips, which enable fast access to large amounts of data. The at least one GPU may have specialized units for specific tasks.

CPUs may be provided for carrying out central control tasks and for a coordination of the various processing steps. They may implement and provide efficient sequential controls, so that the various processing steps are carried out smoothly and in the right order. CPUs are especially efficient for processing steps that require sequential and serial processing, such as the step-by-step analysis of image data or the execution of algorithms that cannot be executed in parallel. CPUs may further execute complex algorithms and models, which make possible a precise and detailed analysis of image data. This may, for example, include an image registration, modeling, and/or simulations.

GPUs may efficiently pre-process and filter image data, in that they carry out all operations such as noise suppression edge detection and/or contrast enhancement in real time. These processing steps may profit from the parallel processing capacity of the GPUs. In a reconstruction of 3D images from 2D slice images, such as during CT or MRT scans for example, GPUs may carry out the extensive calculations in parallel, which leads to a significant speeding up of the process. GPUs may further be used for carrying out tasks in the area of machine learning and/or pattern recognition. In this case, the GPUs may, for example, analyze large amounts of image data in parallel and/or train complex neural networks.

The at least one CPU may further take over control and coordination, while the at least one GPU is carrying out the parallel data processing and/or analysis.

The form of embodiment may make possible an especially efficient (e.g., resource-efficient) processing of the medical image data.

In a further form of embodiment of the method, a duration of the processing of the medical image data may be captured in step b.1). The provision of the signal in step b.2) may be adapted as a function of the last processing duration captured.

The processing duration may include the period of time that is needed for the complete processing of the medical image data in step b.1). The capture of the processing duration may, for example, be undertaken by the processing unit or by a further sensor. The provision of the signal in step b.2) may be undertaken as a function of the last processing duration captured in each case. For example, the signal may be provided in step b.2), depending on the last processing duration captured, such that the processing duration may be taken into account (e.g., compensated for) when executing the repeated carrying out of the acts a) to c).

This provides that the carrying out of the acts a) to c) may be dynamically adapted to the currently captured processing duration in order to provide an optimal efficiency and accuracy.

In a further form of embodiment of the method, in act b.1), a respective processing duration of one or more processing steps of the number of processing steps may be captured. The provision of the signal may be adapted depending on the at least one processing duration last captured.

The number of processing steps may have, at least partly (e.g., completely) different processing durations. The respective processing duration may in this case include a period of time from beginning to end of the respective processing step.

In one embodiment, the processing duration of a predefined processing step of the number of processing steps may be captured. As an alternative, a respective processing duration of the number of processing steps may be captured. The capture of the at least one processing duration may be undertaken by the processing unit or by a further sensor.

The provision of the signal in act b.2) may be carried out depending on the respective at least one processing duration last captured. For example, the signal may be provided in act b.2) depending on the last at least one processing duration captured such that the at least one processing duration may be taken in account (e.g., compensated for) when executing the repeated carrying out of the acts a) to c).

This provides that the carrying out of the acts a) to c) may be adapted dynamically to the at least one processing duration currently captured, in order to provide an optimal efficiency and accuracy.

In a further form of embodiment of the method, act a) may include a pre-processing of the medical image data with a static pre-processing duration.

The pre-processing of the medical image data may be undertaken after the provision of the medical image data by the provision unit and before the processing of the medical image data in act b.1). The pre-processing of the medical image data may be undertaken within a predefined (e.g., static) period of time (e.g., independently of a complexity and/or amount of the medical image data). In this case, the static pre-processing duration may include the period of time from a beginning to and end of the pre-processing.

The pre-processing may, for example, include a noise suppression, segmentation, filtering, feature extraction, and/or amplification of the medical image data. In one embodiment, the pre-processing of the medical image data may be undertaken by the processing unit.

The form of embodiment may make possible an improved provision of the processed medical image data.

In a further form of embodiment of the method, a region of interest of an examination object depicted in the medical image data may be predetermined and/or be identified based on the medical image data. The provision of the signal in act b.2) may be undertaken when a predefined processing state of the region of interest depicted in the medical image data is reached.

The region of interest (ROI) may include a spatial region (e.g., a volume) within the examination object, which is depicted in the medical image data. The predetermination of the region of interest may, for example, be undertaken based on a user input (e.g., an annotation). The user input may, for example, be captured by an input unit. The user input may, for example, be captured with regard to a graphical representation of the unprocessed or the graphical representation of the processed medical image data.

As an alternative or in addition, the region of interest may be identified based on the medical image data. The identification of the region of interest may, for example, include an identification of geometrical and/or anatomical features in the medical image data.

In one embodiment, the provision of the signal in act b.2) may be undertaken when the predefined processing state is reached (e.g., on conclusion of a predefined processing step) of the region of interest depicted in the medical image data. A processing still outstanding of the remaining regions of the medical image data outside of the region of interest need not be taken into consideration for the provision of the signal in act b.2).

The form of embodiment may make possible an especially time-efficient provision of the graphical representation of the processed image data (e.g., of the region of interest in the medical image data).

In a further form of embodiment of the method, there may be a buffering of the medical image data between act a) and act b). The predefined processing stage may be predetermined such that the buffering of the medical image data is minimized.

The buffering of the medical image data between the acts a) and b) may include a temporary storage (e.g., an intermediate storage) of the medical image data (e.g., for a few milliseconds). In one embodiment, the buffered (e.g., intermediately stored) medical image data may be provided to the processing unit as soon as the unit is ready for the processing of the medical image data (e.g., as soon as the respective ongoing processing of the previous medical image data has reached a predefined processing stage, such as a predefined processing step).

In one embodiment, the predefined processing stage (e.g., the predefined processing step) may be predetermined, such that the buffering of the medical image data is minimized. In this case, the medical image data may be intermediately stored only for as long as is absolutely necessary in order to provide a continuous and uninterrupted provision for the following processing steps. In this way, delays will be avoided, and the image processing unit will always be supplied with sufficient image data, even in scenarios with high data rate and variable processing time.

The present embodiments relate, in a second aspect, to a system for optimizing provision of medical image data. The system includes a provision unit, a processing unit, and a receive unit. The system is configured to carry out a method of the present embodiments for optimizing provision of medical image data.

The advantages of the Systems essentially correspond to the advantages of the method. Features, advantages, or alternative forms of embodiment may likewise also be transferred to the other subject matter and vice versa.

In a further form of embodiment of the system, the system may also include a medical imaging device that is configured to record the medical image data.

The medical imaging device may, for example, include a Magnetic Resonance Tomography (MRT) system and/or a Computed Tomography (CT) system and/or a medical x-ray device (e.g., a medical C-arm x-ray device), and/or an ultrasound device, and/or a Positron Emission Tomography (PET) system, and/or an optical camera (e.g., in an endoscope), and/or an operation microscope, and/or an Optical Coherence Tomography (OCT).

The present embodiments relate, in a third aspect, to a computer program product including a computer program (e.g., having machine-readable instructions), which is able to be loaded directly into a memory of a processing unit. The computer program includes program sections for carrying out all acts of a method of the present embodiments for processing medical image data when the program sections are executed by the processing unit.

In one embodiment, the computer program product may, for example, include software with a source code that still has to be compiled and linked or only has to be interpreted, or an executable software code that is yet to be loaded into the processing unit for execution. The computer program product enables the method for optimizing provision of medical image data to be executed quickly, identically repeatable, and robustly by a processing unit. The computer program product is configured so that it may carry out the method steps of the present embodiments by the processing unit. The provision unit and the receive unit may, for example, be mapped in software in the computer program product.

The computer program product is, for example, stored on a computer-readable memory medium or held on a network or server; from here, it may be loaded into the processor of a processing unit that is directly connected to the processing unit or may be embodied as part of the processing unit. Further, control information of the computer program product may be stored on an electronically-readable data medium. The control information of the electronically-readable data medium may be configured such that it carries out a method of the present embodiments when the data medium is used in a processing unit. Examples of electronically-readable data media are a DVD, a magnetic tape, or a USB stick, on which electronically-readable control information (e.g., software) is stored. When this control information is read from the data medium and stored in a processing unit, all forms of embodiment of the previously described method may be carried out.

A largely software-based realization has the advantage that processing units already used may be upgraded in a simple way by a software update in order to work in the way of the present embodiments. Such a computer program product, as well as the computer program, may include additional elements such as, for example, documentation and/or additional components, as well as hardware components, such as, for example, hardware keys (e.g., dongles, etc.) for use of the software.

### BRIEF DESCRIPTION OF THE DRAWINGS

Example embodiments are shown in the drawings and will be explained in greater detail below. The same reference characters are used for the same features in different figures.

FIG. 1 shows a schematic diagram of a form of embodiment of a method for processing medical image data;

FIG. 2 shows a schematic diagram of a temporal flow diagram of a form of embodiment of a method for processing medical image data;

FIGS. 3 to 8 show schematic diagrams of various forms of embodiment of a method for processing medical image data; and

FIGS. 9 to 11 show schematic diagrams of various forms of embodiment of a system for processing medical image data.

### DETAILED DESCRIPTION

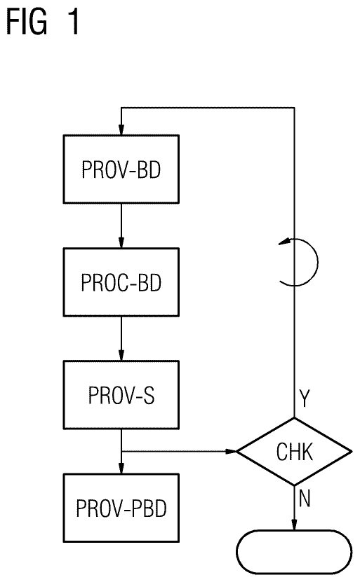

FIG. 1 shows a schematic diagram of a form of embodiment of a method for processing medical image data. In a first act a), medical image data of an examination object may be provided PROV-BD by a provision unit to a processing unit. For example, act a) may include a recording of the medical image data using a medical imaging device. In this case, the medical imaging device may include the provision unit. In a further act b.1), the medical image data may be processed PROC-BD by a processing unit. In a further act b.2), a signal may be provided, PROV-S, by the processing unit to the provision unit on reaching a predefined processing state of the medical image data. In a further act c), the processed medical image data may be provided PROV-PBD by the processing unit to a receive unit. Act c) may include a provision (e.g., a display) of a graphical representation of the processed image data by a display unit. A check, CHK, may further be made as to whether the signal is present. If it is, Y (e.g., with the presence of the signal), the acts a) to c) may be carried out repeatedly.

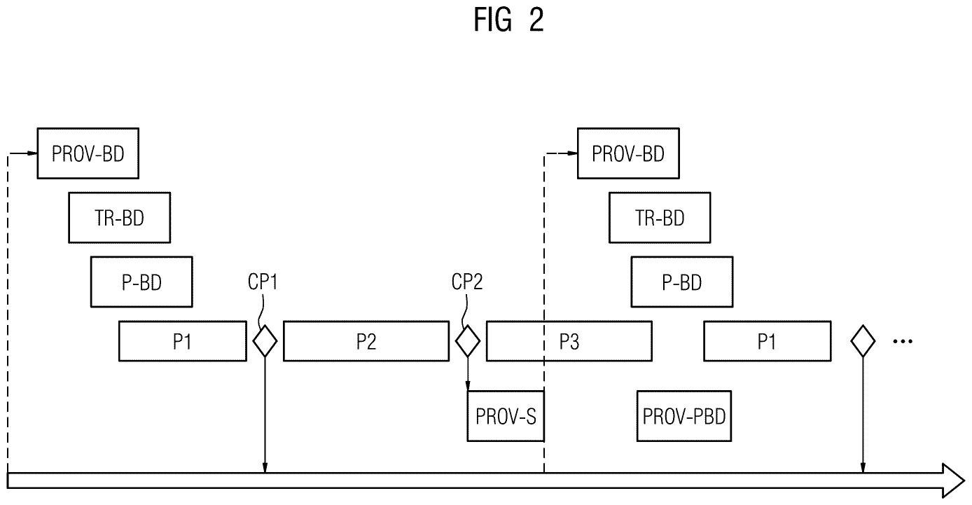

FIG. 2 shows a schematic diagram of a temporal flow diagram of a further form of embodiment of a method for processing medical image data. Illustrated in FIG. 2 is a transmission of the medical image data TR-BD from the provision unit to the processing unit. In this case, act b.1) may include a number of processing acts P1, P2 and P3 for processing PROC-BD of the medical image data, which will be carried out one after the other. In this case, the signal may be sent in act b.2) depending on a predefined processing act of the number of processing acts being reached or ended. For example, checkpoints CP1 and CP2 may be used during the processing of the medical image data in order to check that a respective processing step has been reached. In this case, the signal may be provided, PROV-S, for example, after the second checkpoint CP2 has been reached. The repeated provision of the image data PROV-BD with the presence of the signal may, for example, be undertaken temporally in parallel to other processing of the medical image data (e.g., the third processing step P3).

The processing unit may further include at least one GPU and at least one CPU, which are configured in each case to carry out at least one of the number of processing steps. In such cases, in act b.1), a respective processing duration of one or more processing steps of the number of processing steps may be captured. In one embodiment, the provision of the signal PROV-S may be configured depending on the at least one processing duration last captured (e.g., on a sum of the processing durations of the number of processing steps).

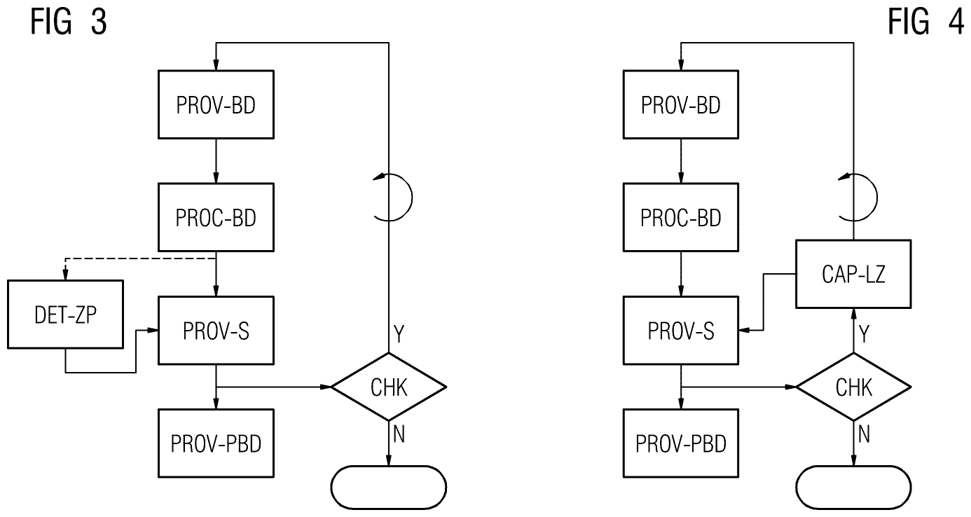

FIG. 3 shows a schematic diagram of a further form of embodiment of a method for processing medical image data. In this case, a future provision time for repeated execution of act c) may be detected DET-ZP. In this case, the provision of the signal PROV-S in act b.2) may additionally be undertaken depending on the future provision time. For example, the future provision time may be detected, DET-ZP, based on: a feature that is identified in the medical image data in act b.1); a change that is identified in the medical image data in step b.1); and/or a latency time of a robotic control of a medical object on or in the examination object. The robotic control is based on the graphical representation of the processed image data.

FIG. 4 shows a schematic diagram of a further form of embodiment of a method for processing medical image data. In this case, a latency time between act b.1) and act c) may be captured, CAP-LZ. The provision of the signal PROV-S in act b.2) may further additionally be undertaken depending on the last latency time captured.

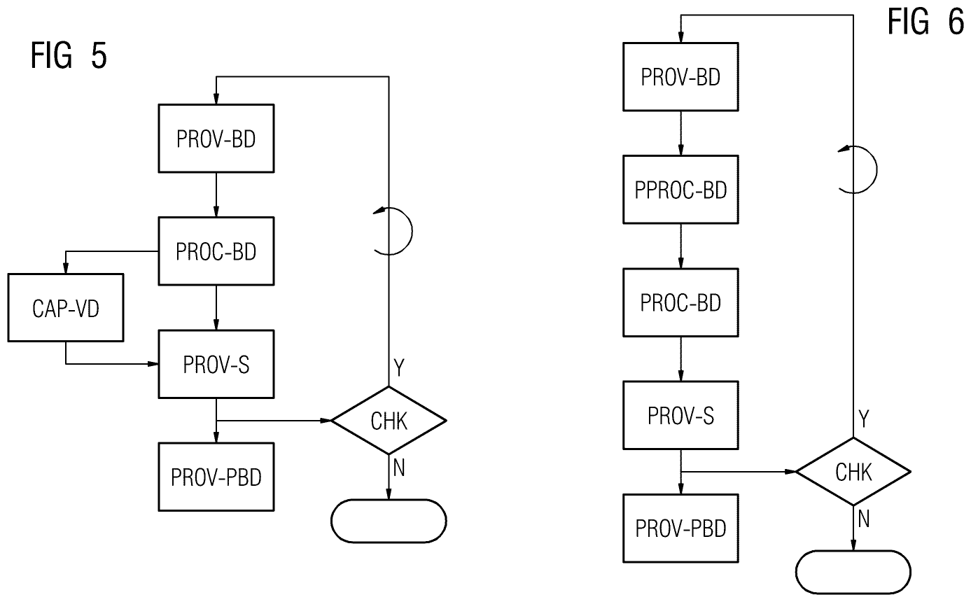

FIG. 5 shows a schematic diagram of a further form of embodiment of a method for processing medical image data. In this case, a processing duration of the processing of the medical image data in act b.1) may be captured, CAP-VD. In one embodiment, the provision of the signal, PROV-S, in act b.2), may be adapted depending on the last processing duration captured.

FIG. 6 shows a schematic diagram a further form of embodiment of a method for processing medical image data. In this case, act a) may include a pre-processing of the medical image data PPROC-BD with a static pre-processing duration.

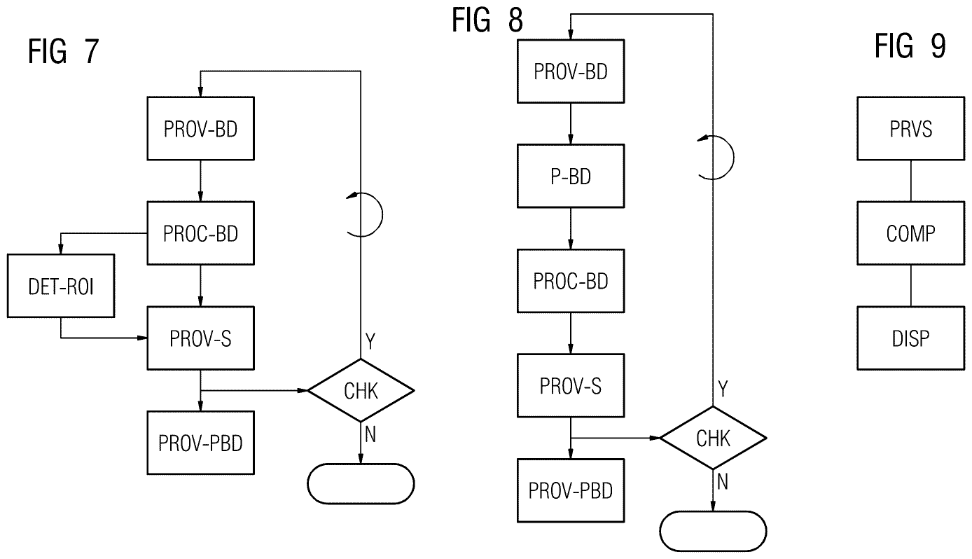

FIG. 7 shows a schematic diagram of a further form of embodiment of a method for processing medical image data. In this case, a region of interest of an examination object depicted in the medical image data and/or based on the medical image data may be detected, DET-ROI. Further, the provision of the signal PROV-S in act b.2) may be undertaken on reaching a predefined processing state of the region of interest depicted in the medical image data. A latency time for a provision of the region of interest depicted in the medical image data may be optimized for the processing unit (e.g., a provision to a predefined processing step and/or processing algorithm). The region of interest may, for example, include a spatial volume, in which a tip of a robotic catheter is arranged. This provides that a few parts of the image data (e.g., an upper left corner), where a transmission or the processing unit may begin, have a non-optimal latency time, but the parts of the image data that depict the region of interest have an optimal latency. Complete image data may be transmitted in a usual order, where the region of interest arrives just at the right time depending on the processing of the depiction of the region of interest.

FIG. 8 shows a schematic diagram of a further form of embodiment of a method depending on the processing of medical image data. In this case, there may be a buffering of the medical image data P-BD between act a) and act b). For example, the medical image data may be placed in an image queue after act a). The predefined processing stage may further be predefined such that the buffering of the medical image data is minimized.

FIG. 9 shows a schematic diagram of a form of embodiment of a system depending on the processing of medical image data. The system may include a provision unit PRVS, a computing unit COMP, and a receive unit (e.g., a display unit DISP). In this case, the system may be configured for carrying out a method for optimizing provision of medical image data. For example, the provision unit PRVS may be configured to provide PROV-BD the medical image data of the examination object to the computing unit COMP. The computing unit COMP may further be configured to process PROC-BD the medical image data. The computing unit COMP may further be configured to provide, PROV-S, the signal to the provision unit PRVS on reaching a predefined processing state of the medical image data, Further, the computing unit COMP may be configured to provide PROV-PBD the processed medical image data to the display unit DISP. The display unit DISP may, for example, include a monitor and/or a display and/or a projector. The display unit DISP may be configured to provide the graphical representation of the processed image data. The system and its respective components may further be configured to repeatedly carry out the acts a) to c) of the method depending on the image processing when the signal is present.

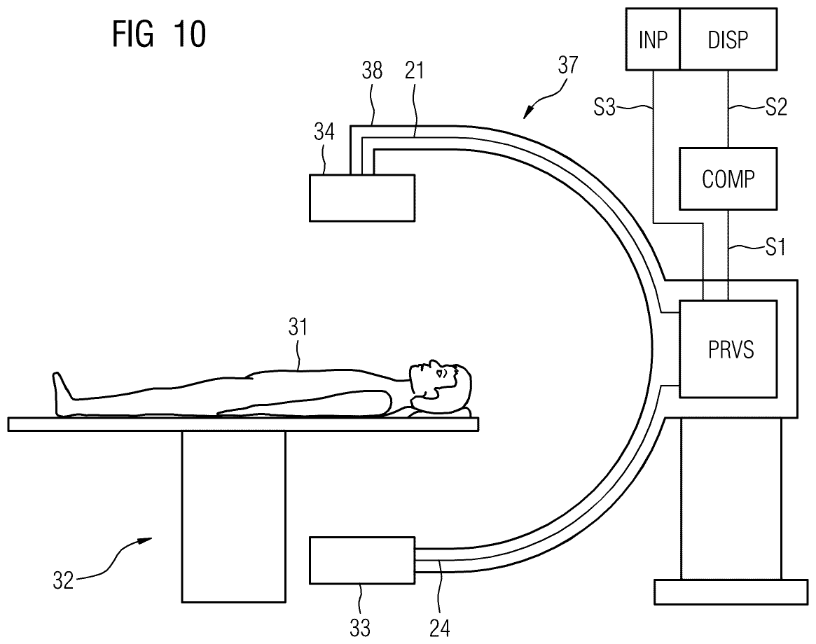

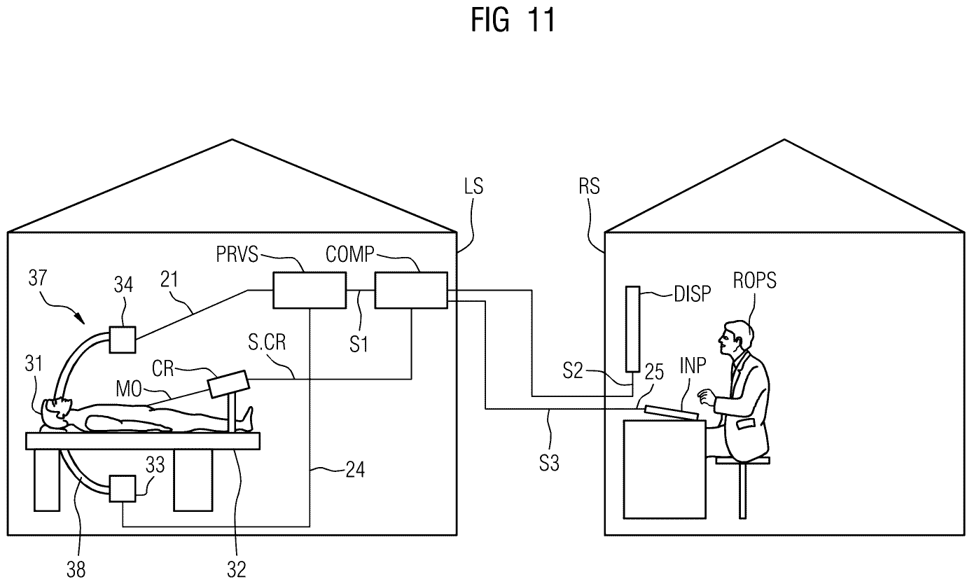

FIG. 10 shows a schematic diagram of a further form of embodiment of a system depending on the processing of medical image data. In this case, the system may include a medical imaging device that is configured for recording the medical image data. In this case, a medical C-arm x-ray device 37 is shown schematically by way of example in FIG. 9 for a medical imaging device for recording of the medical image data. The medical imaging device 37 may have a source 33 (e.g., an x-ray source) and a detector 34 (e.g., an x-ray detector) that are arranged in a defined arrangement on the C arm 38. The C arm 38 may be supported such that they are able to move about one or more axes.

To record the medical image data of the examination object 31 positioned on a patient support apparatus 32, the provision unit PRVS may send a signal 24 to the source 33. Thereafter, the source 33 may emit a radiation bundle (e.g., an x-ray radiation bundle). When the x-ray radiation bundle, after interacting with the examination object 31, arrives at an x-ray-sensitive surface of the detector 34, the detector 34 may send a signal 21 to the provision unit PRVS. The provision unit may capture the medical image data of the examination object 31 (e.g., receive it) with the aid of the signal 21.

The provision unit PRVS may further provide the medical image data to the computing unit COMP using a signal S1. The computing unit COMP may further be configured to provide PROV-PBD the processed medical image data to the display unit DISP using a signal S2. The computing unit COMP may further provide, PROV-S, the signal S1 to the provision unit PRVS when the predefined processing state of the medical image data is reached.

The system may further include an input unit INP (e.g., a keyboard and/or a joystick). The input unit INP may be integrated into the display unit DISP (e.g., with a capacitive and/or resistive input display). The input unit INP may be configured for capture of a user input. The input unit INP may further be configured to provide a signal S3 to the provision unit PRVS depending on the user input captured. The provision unit PRVS may, for example, be configured to control the recording of the medical image data based on the user input. As an alternative or in addition, the provision unit PRVS may be configured to detect the region of interest DET-ROI of the examination object 31 based on the user input.

FIG. 11 shows a schematic diagram of a further form of embodiment of a system for processing medical image data. In this case, the examination object 31, the C-arm x-ray device 37, the provision unit PRVS, and the computing unit COMP may be arranged in an on-site treatment room LS. The input unit INP and the display unit DISP may further be arranged in a remote operating room RS. The input unit INP may be configured to capture the user input of a remote operator ROPS. The display unit DISP may further be configured to provide the graphical representation of the processed medical image data to the remote operators ROPS. The system may further include a movement apparatus CR (e.g., a catheter robot) that may be coupled to the computing unit COMP for communication using a signal S.CR. The movement apparatus CR may be configured for robotic movement of a medical object MO (e.g., of a catheter and/or surgical instrument) that is arranged, in an operating state of the system, at least partly in the examination object 31. In one embodiment, the movement apparatus CR may be able to be controlled for positioning and/or movement of the medical object MO based on the user input of the remote operators ROPS. The computing unit COMP may be coupled to the provision unit PRVS, the movement apparatus CR, the display unit DISP, and/or the input unit INP for signaling (e.g., using a remote transmission channel, such as a network and/or Internet). For a remote transmission channel with varying latency time, the latency time of the remote transmission channel at that moment may be captured (e.g., measured and/or predicted). In this case, the provision of the signal, PROV-S, in act b.2), may additionally be undertaken depending on the last latency time captured in each case.

In one embodiment, the computing unit COMP may be configured to detect, DET-ZP, a future provision time depending on the repeated execution of act c). In this case, the signal may additionally be provided, PROV-S, in act b.2) depending on the future provision time. For example, the future provision time may be detected, DET-ZP, based on a latency time of a robotic control of the medical object MO on or in the examination object 31. In this case, the robotic control may be based on the graphical representation of the processed image data.

The schematic diagrams contained in the figures described do not depict a particular scale or size relationships.

The method described in detail above, as well as the apparatus shown, merely represent example embodiments, which may be modified by the person skilled in the art in a very wide variety of ways, without departing from the area of the invention. Further, the use of the indefinite article “a” or “an” does not exclude the features concerned also being able to be present a number of times. Likewise, the terms “unit” and “element” do not exclude the components involved consisting of a number of interacting sub-components that may, if necessary, also be spatially distributed.

The expression “based on” may be understood in the context of the present application, for example, in the sense of the expression “using.” For example, a formulation according to which a first feature is created (e.g., alternatively, established, determined, etc.) based on a second feature does not exclude that the first feature may be created (e.g., alternatively, established, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for optimizing provision of medical image data, the method comprising:
providing medical image data of an examination object from a provision unit to a computing unit;
processing the medical image data by the computing unit;
providing a signal by the computing unit to the provision unit on reaching a predefined processing state of the medical image data; and
providing the processed medical image data by the computing unit to a receive unit,
wherein on receipt of the signal by the provision unit, the providing of the medical image data, the processing, the providing of the signal, and the providing of the processed medical image data are carried out repeatedly.

2. The method of claim 1, wherein providing the medical image data comprises a recording of the medical image data by a medical imaging device, and
wherein the medical imaging device comprises the provision unit.

3. The method of claim 1, further comprising detecting a future provision time depending on the repeated carrying out of the providing of the processed medical image data,
wherein the providing of the signal is additionally undertaken as a function of the future provision time.

4. The method of claim 3, wherein the future provision time is detected based on:
a feature that is identified in the medical image data in the processing of the medical image data;
a change that is identified in the medical image data in the processing of the medical image data;
a latency time of a robotic control; or
any combination thereof, and
wherein the robotic control is based on the processed image data.

5. The method of claim 1, further comprising capturing a latency time between the processing of the medical image data and the providing of the signal, and
wherein the providing of the signal is additionally undertaken as a function of the latency time last captured.

6. The method of claim 1, wherein the processing comprises a number of processing steps for the processing of the medical image data that are carried out one after the other, and
wherein the providing of the signal is undertaken as a function of a predefined processing step of the number of processing steps being reached or ended.

7. The method of claim 6, wherein the computing unit comprises at least one GPU and at least one CPU that are configured in each case to carry out at least one of the number of processing steps.

8. The method of claim 6, wherein the processing comprises capturing a processing duration of the processing of the medical image, and
wherein the providing of the signal is adapted as a function of the processing duration last captured.

9. The method of claim 8, wherein the processing comprises capturing a respective processing duration of one or more processing steps of the number of processing steps, and
wherein the providing of the signal is adapted as a function of the at least one processing duration last captured.

10. The method of claim 1, wherein providing the medical image data comprises a pre-processing of the medical image data with a static pre-processing duration.

11. The method of claim 1, wherein a region of interest of an examination object depicted in the medical image data is predetermined, is detected based on the medical image data, or a combination thereof, and
wherein the providing of the signal is undertaken when a predefined processing state of the region of interest depicted in the medical image data is reached.

12. The method of claim 1, further comprising buffering the medical image data between the providing of the medical image data and the processing of the medical image data,
wherein the predefined processing state is predetermined such that the buffering of the medical image data is minimized.

13. A system for optimizing provision of medical image data, the system comprising: a provision unit;
a computing unit; and
a receive unit,
wherein the system is configured for optimization of provision of medical image data, the optimization comprising:
provision of medical image data of an examination object from a provision unit to a computing unit;
process of the medical image data by the computing unit;
provision of a signal by the computing unit to the provision unit on reaching a predefined processing state of the medical image data; and
provision of the processed medical image data by the computing unit to a receive unit,
wherein on receipt of the signal by the provision unit, the provision of the medical image data, the process, the provision of the signal, and the provision of the processed medical image data are carried out repeatedly.

14. The system of claim 13, further comprising a medical imaging device that is configured for recording of the medical image data.

15. In a non-transitory computer-readable storage medium that stores instructions executable by one or more processors to optimize provision of medical image data, the instructions comprising:
providing medical image data of an examination object from a provision unit to a computing unit;
processing the medical image data by the computing unit;
providing a signal by the computing unit to the provision unit on reaching a predefined processing state of the medical image data; and
providing the processed medical image data by the computing unit to a receive unit,
wherein on receipt of the signal by the provision unit, the providing of the medical image data, the processing, the providing of the signal, and the providing of the processed medical image data are carried out repeatedly.
